(19) United States
(12) Patent Application Publication
(10) Pub. No.: US 2022/0315866 A1
(43) Pub. Date: Oct. 6, 2022

(54) Detergent Composition

(71) Applicant: Novozymes A/S, Bagsvaerd (DK)

(72) Inventors: Morten Foverskov, Herlev (DK); Mette Bruni Let, Lyngby (DK)

(73) Assignee: Novozymes A/S, Bagsvaerd (DK)

(21) Appl. No.: 17/642,974

(22) PCT Filed: Sep. 18, 2020
(86) PCT No.: PCT/EP2020/076069

§ 371 Date: Mar. 15, 2022

(30) Foreign Application Priority Data
Sep. 19, 2019 (EP) 19198286.7

(51) Int. Cl. C11D 9/40; C11D 10/04

(52) U.S. Cl. CPC C11D 9/40; C11D 10/04

(57) ABSTRACT

The present invention relates to detergent compositions comprising alpha amylase mixtures. The compositions of the invention are suitable as e.g. cleaning or detergent compositions, such as laundry detergent compositions and dish wash compositions, including automatic dish wash compositions.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to novel compositions comprising alpha amylase mixtures. The compositions of the invention are suitable as e.g. cleaning or detergent compositions, such as laundry detergent compositions and dish wash compositions, including automatic dish wash compositions.

### Description of the Related Art

Alpha-amylases (α-1,4-glucan-4-glucanohydrolases, E.C. 3.2.1.) are a group of enzymes that hydrolyzes starch, glycogen, and other related polysaccharides by cleaving the internal α-1,4-glucosidic bonds. It has been used for many years been in, e.g., laundry where is it well-known that alpha-amylases have a beneficial effect in removal of starch containing, or starch-based, stains. However, in other commercial applications the enzyme has become important, such as in the initial stages (liquefaction) of starch processing, in textile desizing, in alcohol production and as cleaning agents in detergent compositions.

In recent years there has been a desire to improve the properties of various amylases. In particular, the object of reducing the temperature of the laundry in order to reduce the energy consumption has been of primary focus when referring to the household care sector. Thus, many efforts have been put into finding improved alpha-amylase variants.

To improve the cost and/or the performance of enzymes there is an ongoing search for enzymes with altered properties, such as increased activity at low temperatures, increased stability, increased specific activity at a given pH, altered Ca²⁺ dependency, increased stability in the presence of other detergent ingredients (e.g. bleach, surfactants etc.) etc.

Detergent compositions have been described, but there is a continued need for improved detergent compositions, wherein the enzymes within the detergent compositions are able to act synergistically in cleaning performance. Thus, it is an objective of the present invention to provide such detergent compositions.

### SUMMARY OF THE INVENTION

The present invention relates to a detergent composition comprising an alpha-amylase, the alpha-amylase is a mixture of at least two alpha-amylases.

The present invention relates also to a method of dishwashing comprising with a detergent composition according to the invention.

The present invention relates also to a method of laundering comprising laundering a fabric with a detergent composition according to the invention.

### Definitions

The term “alpha-amylase” means an alpha-amylase having alpha-amylase activity, i.e. the activity of alpha-1,4-glucan-4-glucanohydrolases, E.C. 3.2.1.1, which constitute a group of enzymes, catalysing hydrolysis of starch and other linear and branched 1,4-glucosidic oligo- and polysaccharides.

The term “wild-type alpha-amylase” means an alpha-amylase as expressed by a naturally occurring microorganism, such as a bacterium, yeast, or filamentous fungus found in nature.

The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

The term “operably linked” means a configuration in which a control sequence is placed at an appropriate position relative to the coding sequence of a polynucleotide such that the control sequence directs the expression of the coding sequence.

The term “fragment” means a polypeptide having one or more (e.g., several) amino acids absent from the amino and/or carboxyl terminus of a mature polypeptide or domain; wherein the fragment has alpha-amylase activity.

The term “control sequences” means all components necessary for the expression of a polynucleotide encoding an alpha-amylase of the present invention. Each control sequence may be native or foreign to the polynucleotide encoding the variant or native or foreign to each other.

Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding an alpha-amylase.

The term “expression” includes any step involved in the production of the or alpha-amylase including, but not limited to, transcription, post-transcriptional modification, translation, post-translational modification, and secretion.

The term “expression vector” means a linear or circular DNA molecule that comprises a polynucleotide encoding an alpha-amylase and is operably linked to additional nucleotides that provide for its expression.

The term “transcription promoter” is used for a promoter which is a region of DNA that facilitates the transcription of a particular gene. Transcription promoters are typically located near the genes they regulate, on the same strand and upstream (towards the 5′ region of the sense strand).

The term “transcription terminator” is used for a section of the genetic sequence that marks the end of gene or operon on genomic DNA for transcription.

The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

It is within the knowledge of the skilled person to know how to align amino acid sequences in order to determine which amino acid in a particular position referred to herein “corresponds to” another amino acid sequence not listed herein. Thus, the term “position corresponding to” as used herein, is well-known within the art. The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) is used as the percent identity and is calculated as follows:

(Identical Residues×100)/(Length of Alignment−Total Number of Gaps in Alignment)

The term “improved wash performance” is defined herein as a detergent composition displaying an increased wash performance relative to the wash performance of a similar detergent composition without an alpha-amylase alone. Thus, it is believed that the detergent composition comprising at least two alpha-amylases has a beneficial effect on wash performance. The detergent composition comprising at least two alpha-amylase may show synergy and thereby provide a detergent composition having an even further improved wash performance when compared to a detergent composition comprising only one alpha-amylase.

The term “wash performance” includes wash performance in laundry but also e.g. in dish wash. The wash performance may be quantified as described under the definition of “wash performance” herein. It will be appreciated by persons skilled in the art that the enhanced wash performance may be achieved under only some or perhaps all wash conditions and/or with or without the presence of bleach.

The term “wash cycle” is defined herein with respect to dishwashing as a washing operation wherein dishware are exposed to the wash liquor for a period of time by circulating the wash liquor and spraying the wash liquor onto the dishware in order to clean the dishware and finally the superfluous wash liquor is removed. A wash cycle may be repeated one, two, three, four, five or even six times at the same or at different temperatures. Hereafter the dishware is generally rinsed and dried. One of the wash cycles can be a soaking step, where the dishware is left soaking in the wash liquor for a period.

The term “wash liquor” is defined herein as the solution or mixture of water and detergent components.

The term “wash performance” with respect to automatic dishwashing is defined herein as the ability of an automatic dishwashing detergent composition to remove soil present on dishware to be cleaned during washing. The wash performance may be measured by inspecting the washed dishware, light reflectance (460 nm) or by measuring weight to determine how much of the soil has been removed. This can be done by measuring the difference in weight on plates, tiles or similar. Wash performance may be determined in automatic dishwashing as described in Examples section.

The term “wash time” with respect to automatic dishwashing is defined herein as the time it takes for the entire washing process; i.e. the time for the wash cycle(s) and rinse cycle(s) together.

The term “detergent composition”, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, soap bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types.

The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”).

The term “automatic dishwashing detergent composition” refers to compositions comprising detergent components, which composition is intended for cleaning dishware such as plates, cups, glasses, bowls, cutlery such as spoons, knives, forks, serving utensils, ceramics, plastics, metals, china, glass and acrylics in a dishwashing machine. It is not intended that the present invention be limited to any particular detergent formulation or composition.

The term “detergent composition” is not intended to be limited to compositions that contain surfactants. It is intended that in addition to the enzymes herein described, the detergents compositions may comprise, e.g. one or more additional components selected from stabilizing agents, surfactants, hydrotopes, builders, co-builders, chelating agents, bleaching systems, bleach activators, polymers and fabric-hueing agents.

The term “fabric” encompasses any textile material. Thus, it is intended that the term encompass garments, as well as fabrics, yarns, fibres, non-woven materials, natural materials, synthetic materials, and any other textile material.

The term “textile” refers to woven fabrics, as well as staple fibres and filaments suitable for conversion to or use as yarns, woven, knit, and non-woven fabrics. The term encompasses yarns made from natural, as well as synthetic (e.g., manufactured) fibres. The term, “textile materials” is a general term for fibres, yarn intermediates, yarn, fabrics, and products made from fabrics (e.g., garments and other articles).

The term “non-fabric detergent compositions” include non-textile surface detergent compositions, including but not limited to compositions for hard surface cleaning, such as dishwashing detergent compositions, oral detergent compositions, denture detergent compositions, and personal cleansing compositions.

The term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like.

The term “effective amount” of an enzyme refers to the quantity of enzyme described hereinbefore that achieves a desired level of enzymatic activity, e.g., in a defined detergent composition.

The term “water hardness” or “degree of hardness” or “dH” or “° dH” as used herein refers to German degrees of hardness. One degree is defined as 10 milligrams of calcium oxide per litre of water.

The term “relevant washing conditions” is used herein to indicate the conditions, particularly washing temperature, time, washing mechanics, detergent concentration, type of detergent and water hardness, actually used in households in a detergent market segment.

The term “adjunct materials” means any liquid, solid or gaseous material selected for the particular type of detergent composition desired and the form of the product (e.g., liquid, granule, powder, bar, paste, spray, tablet, gel, or foam composition), which materials are also preferably compatible with the enzymes used in the composition. In some embodiments, granular compositions are in “compact” form, while in other embodiments, the liquid compositions are in a “concentrated” form.

The term “stain removing enzyme” as used herein, describes an enzyme that aids the removal of a stain or soil from a fabric or a hard surface. Stain removing enzymes act on specific substrates, e.g., protease on protein, amylase on starch, lipase and cutinase on lipids (fats and oils), pectinase on pectin and hemicellulases on hemicellulose. Stains are often depositions of complex mixtures of different components which either results in a local discolouration of the material by itself or which leaves a sticky surface on the object which may attract soils dissolved in the washing liquor thereby resulting in discolouration of the stained area. When an enzyme acts on its specific substrate present in a stain the enzyme degrades or partially degrades its substrate thereby aiding the removal of soils and stain components associated with the substrate during the washing process. For example, when a protease acts on a grass stain it degrades the protein components in the grass and allows the green/brown colour to be released during washing.

The term “Sequence identity” The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

The term “subsequence” means a polynucleotide having one or more (e.g., several) nucleotides absent from the 5′ and/or 3′ end of a mature polypeptide coding sequence; wherein the subsequence encodes a fragment having protease activity.

The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### DETAILED DESCRIPTION OF THE INVENTION

The present invention relates to a detergent composition comprising an alpha-amylase, the alpha-amylase is a mixture of at least two alpha-amylases.

The one aspect, the present invention relates to a detergent composition comprising an alpha-amylase, wherein the alpha-amylase is a mixture of at least three alpha-amylases.

The one aspect, the present invention relates to a detergent composition comprising an alpha-amylase, wherein the alpha-amylase is a mixture of at least four alpha-amylases.

The one aspect, the present invention relates to a detergent composition comprising an alpha-amylase, wherein the alpha-amylase is a mixture of at least five alpha-amylases.

In one aspect, the detergent composition comprising the alpha-amylase is selected from the group consisting of:
a. variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering);
b. variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering).
c. variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering);
d. variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering);
e. variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering);
f. variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering);
g. a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering),
h. a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering),
i. a variant of a parent amylase, said variant amylase or parent amylase has at least 60%, at least 65%, at least 70%, at least 75%, at least, 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering), and
j. variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the alpha-amylase may be added to a detergent composition in an amount corresponding to 0.001-100 mg of protein, such as 0.001-100 mg of protein, preferably 0.001-50 mg of protein, more preferably 0.001-25 mg of protein, even more preferably 0.001-10 mg of protein per gram of the composition.

In one aspect, the detergent composition is a phosphate-free composition.

In one aspect, the detergent composition comprises 0.001% to 95% alpha-amylase mixture by weight of the composition.

In one aspect, the detergent composition comprises amylase in the range from 0.001 to 10 mg per gram of the composition.

In one aspect, the detergent composition has a pH in the range of 2.0-12.0, such as 3.0-11.8, such as 4-11.5, such as 5-11.

In one aspect, the detergent composition has improved wash performance compared to said reference alpha-amylase.

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering) and variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, optionally 70%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 2 and further comprising a mutation at one or more positions corresponding to position: 51, 246 or 334 and the position 202 comprises a methionine (using SEQ ID NO: 2 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 3 and optionally at least one mutation at an amino acid corresponding to 181, 182, 183 and 184 (using SEQ ID NO: 3 for numbering) and a variant of a parent amylase, said variant amylase or parent amylase has at least 60%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering) and a variant of a parent amylase, said variant amylase or parent amylase has at least 60 at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 5 and further comprising a mutation at one or more positions corresponding to position: 405, 421, 422, or 428 (using SEQ ID NO: 5 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60 at least 65%, at least 70%, at least 75%, at least 80%, at least 85%, at least 90%, at least 95% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the detergent composition comprises an alpha-amylase mixture of a variant of a parent amylase, said variant amylase or parent amylase has at least 60%, at least 65%, at least 70%, at least 75%, at least 80%, at least 85%, at least 90%, at least 95% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering) and a variant of a parent amylase, said variant amylase or parent amylase has at least 60%, at least 65%, at least 70%, at least 75%, at least 80%, at least 85%, at least 90%, at least 95% sequence identity to SEQ ID NO: 10 and further comprising a mutation at one or more positions corresponding to position 74, 172, 190, 202, 208, 255, 261, or 365 (using SEQ ID NO: 10 for numbering).

In one aspect, the invention is directed to detergent compositions comprising alpha-amylase mixture of the present invention in combination with one or more additional cleaning composition components. The choice of additional components is within the skill of the artisan and includes conventional ingredients, including the exemplary non-limiting components set forth below.

In one aspect, the invention is directed to an ADW (Automatic Dish Wash) compositions comprising an alpha-amylase mixture as disclosed in combination with one or more additional ADW composition components. The choice of additional components is within the skill of the artisan and includes conventional ingredients, including the exemplary non-limiting components set forth below.

In one aspect the invention is directed to a laundry detergent composition comprising an alpha-amylase mixture as disclosed in combination with one or more additional laundry detergent composition components. The choice of additional component is within the skills of the artisan and include conventional ingredients, including the exemplary non-limiting components set forth below.

### Enzymes

The detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

### Additional Enzymes

The composition of the invention may further comprise one or more additional enzymes which provide cleaning and/or wash performance. Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, ß-glucanases, arabinosidases, nucleases, hyaluronidase, chondroitinase, laccase, chlorophyllases, other amylases, or mixtures thereof. A typical combination is an enzyme cocktail that may comprise e.g. alpha-amylase and protease in conjunction with a mannanase. When present in a composition, the aforementioned additional enzymes may be present at levels from 0.00001 to 2 wt %, from 0.0001 to 1 wt % or from 0.001 to 0.5 wt % enzyme protein by weight of the active components in the composition.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

In one aspect preferred enzymes include a cellulase Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme Premium®, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

### Mannanases

In one aspect preferred enzymes include a mannanase. Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Peroxidases/Oxidases

In one aspect preferred enzymes include a peroxidase. A suitable peroxidase is preferably a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

Suitable peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Nucleases

In one aspect preferred enzymes include a nucleases. Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a fungi or bacterium. In particular, a DNase which is obtainable from a species of Bacillus is preferred; in particular a DNase which is obtainable from Bacillus cibi, Bacillus subtilis or Bacillus licheniformis is preferred. Examples of such DNases are described in WO 2011/098579, WO2014/087011 and WO2017/060475. Particularly preferred is also a DNase obtainable from a species of Aspergillus; in particular a DNase which is obtainable from Aspergillus oryzae, such as a DNase described in WO 2015/155350. The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates as described above, liquids, in particular stabilized liquids, or slurries.

### Proteases

In one aspect preferred enzymes include a protease. Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Examples of preferred proteases include:

(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V205I, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;

(b) a variant of the polypeptide of SEQ ID NO: 1 of WO 2016/001449 with the mutation S99SE, wherein position numbers are based on the numbering of SEQ ID NO: 2 of WO 2016/001449;

(c) a variant of the polypeptide of SEQ ID NO: 1 of WO 2016/001449 with the mutation S99AD, wherein position numbers are based on the numbering of SEQ ID NO: 2 of WO 2016/001449;

(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;

(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;

(f) a variant of the polypeptide of SEQ ID NO: 1 of WO 2016/001449 with the substitutions S9R+A15T+G61E+V68A+A194P+V205I+Q245R+N261D, wherein position numbers are based on the numbering of SEQ ID NO: 2 of WO 2016/001449;

(g) a variant of the polypeptide of SEQ ID NO: 1 of WO 2016/001449 with the substitutions S99D+S101R/E+S103A+V104I+G160S; for example a variant of SEQ ID NO: 1 of WO 2016/001449 with the substitutions S3T+V4I+S99D+S101E+S103A+V104I+G160S+V205I, wherein position numbers are based on the numbering of SEQ ID NO: 2 of WO 2016/001449;

(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;

(i) the polypeptide disclosed in GENESEQP under accession number BER84782, corresponding to SEQ ID NO: 302 in WO 2017/210295;

(j) a variant of the polypeptide of SEQ ID NO: 1 of WO 2016/001449 with the substitutions S99D+S101E+S103A+V104I+S156D+G160S+L262E, wherein position numbers are based on the numbering of SEQ ID NO: 2 of WO 2016/001449;

(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;

(l) a variant of the polypeptide of SEQ ID NO: 1 of WO 2016/001449 with the substitutions V68A+S106A, wherein position numbers are based on the numbering of SEQ ID NO: 2 of WO 2016/001449; and

(m) a variant of the polypeptide of SEQ ID NO: 1 of WO 2004/067737 with the substitutions S27K+N109K+S111E+S171E+S173P+G174K+S175P+F180Y+G182A+L184F+Q198E+N199+T297P, wherein position numbers are based on the numbering of SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™ Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Purafect Prime, Preferenz P110™, Effectenz P1000™, Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

In one aspect preferred enzymes include a lipase and/or cutinase. Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases:

In one aspect preferred enzymes include another amylase. Suitable amylases which can be used together with the compositions of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T4595, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T4595+D460T+G476K

T38N+N126Y+T129I+F153W+R178*+G179*+T180D+E187P+1203Y+G476K+G477E wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Amplify Prime™, Atlantic™, Arctic™, Everest™, Duramyl™′ Termamyl™, Fungamyl™, Stainzyme™, Stainzyme PIus™, Natalase™, Liquozyme X and BAN™ (from Novozymes A/S), and Rapidase™, Purastar™/Effectenz™, Powerase, Preferenz S1000, Preferenz S100, Preferenz S110 and Preferenz S210 (from Genencor International Inc./DuPont).

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a surfactant system (comprising more than one surfactant) e.g. a mixture of one or more nonionic surfactants and one or more anionic surfactants. In one embodiment the detergent comprises at least one anionic surfactant than at least one non-ionic surfactant, the weight ratio of anionic to nonionic surfactant may be from 10:1 to 1:10. In one embodiment the amount of anionic surfactant is higher than the amount of non-ionic surfactant e.g. the weight ratio of anionic to non-ionic surfactant may be from 10:1 to 1.1:1 or from 5:1 to 1.5:1. The amount of anionic to non-ionic surfactant may also be equal and the weight ratios 1:1. In one embodiment the amount of non-ionic surfactant is higher than the amount of anionic surfactant and the weight ratio may be 1:10 to 1:1.1. Preferably the weight ratio of anionic to non-ionic surfactant is from 10:1 to 1:10, such as from 5:1 to 1:5, or from 5:1 to 1:1.2. Preferably, the weight fraction of non-ionic surfactant to anionic surfactant is from 0 to 0.5 or 0 to 0.2 thus non-ionic surfactant can be present or absent if the weight fraction is 0, but if non-ionic surfactant is present, then the weight fraction of the nonionic surfactant is preferably at most 50% or at most 20% of the total weight of anionic surfactant and non-ionic surfactant. Light duty detergent usually comprises more nonionic than anionic surfactant and there the fraction of non-ionic surfactant to anionic surfactant is preferably from 0.5 to 0.9. The total weight of surfactant(s) is typically present at a level of from about 0.1% to about 60% by weight, such as about 1% to about 40%, or about 3% to about 20%, or about 3% to about 10%. The surfactant(s) is chosen based on the desired cleaning application, and may include any conventional surfactant(s) known in the art. When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, typically available as sodium or potassium salts or salts of monoethanolamine (MEA, 2-aminoethan-1-ol) or triethanolamine (TEA, 2,2′,2″-nitrilotriethan-1-ol); in particular, linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids. Furthermore, salts of fatty acids (soaps) may be included.

When included therein the detergent will usually contain from about 1% to about 40% by weight of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADM EAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO) e.g. the AEO-series such as AEO-7, alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamine oxides, in particular N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Additional bio-based surfactants may be used e.g. wherein the surfactant is a sugar-based non-ionic surfactant which may be a hexyl-β-D-maltopyranoside, thiomaltopyranoside or a cyclic-maltopyranoside, such as described in EP2516606 B1.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically in the range 40-65%, particularly in the range 50-65%. The builder and/or co-builder may particularly be a chelating agent that forms water-soluble complexes with Ca and Mg. Any builder and/or co-builder known in the art for use in cleaning detergents may be utilized.

Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Clariant), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain from about 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N, N′-disuccinic acid (EDDS), methylglycinediacetic acid (MGDA), glutamic acid-N,N-diacetic acid (GLDA), 1-hydroxyethane-1,1-diylbis(phosphonic acid (HEDP),

ethylenediaminetetramethylenetetrakis(phosphonic acid) (EDTMPA),

diethylenetriaminepentamethylenepentakis(phosphonic acid) (DTMPA or DTPMPA),

N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)aspartic acid (SMAS), N-(2-sulfoethyl)aspartic acid (SEAS), N-(2-sulfomethyl)glutamic acid (SMGL), N-(2-sulfoethyl)glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), aminotrimethylenetris(phosphonic acid) (ATM P), and combinations and salts thereof. Further exemplary builders and/or co-builders are described in, e.g., WO 09/102854, U.S. Pat. No. 5,977,053

### Bleaching Systems

The detergent composition may contain 0-50% by weight, such as 1-40%, such as 1-30%, such as about 1% to about 20%, of a bleaching system. Any oxygen-based bleaching system comprising components known in the art for use in cleaning detergents may be utilized. Suitable bleaching system components include sources of hydrogen peroxide; peracids and sources of peracids (bleach activators); and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.
b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)₂, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

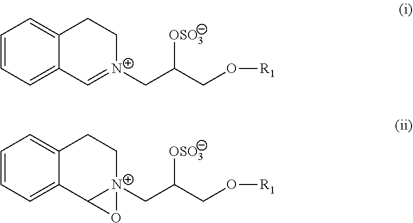

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242.

Suitable photobleaches may for example be sulfonated zinc or aluminium phthalocyanines.

### Polymers

The detergent composition may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties. Exemplary polymers include (carboxymethyl)cellulose (CMC), poly(vinyl alcohol) (PVA), poly(ethyleneglycol) or poly(ethylene oxide) (PEG or PEO), ethoxylated poly(ethyleneimine), (carboxymethyl)inulin (CMI), carboxylate polymers and polycarboxylates such as polyacrylates, maleic/acrylic acid copolymers, acrylate/styrene copolymers, poly(aspartic) acid, and lauryl methacrylate/acrylic acid copolymers, hydrophobically modified CMC (HM-CMC), silicones, copolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Particularly preferred polymer is ethoxylated homopolymer Sokalan® HP 20 from BASF, which helps to prevent redeposition of soil in the wash liquor. Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

### Microorganisms

The detergent composition may also comprise one or more microorganisms, such as one or more fungi, yeast, or bacteria.

In an embodiment, the one or more microorganisms are dehydrated (for example by lyophilization) bacteria or yeast, such as a strain of Lactobacillus.

In another embodiment, the microorganisms are one or more microbial spores (as opposed to vegetative cells), such as bacterial spores; or fungal spores, conidia, hypha. Preferably, the one or more spores are Bacillus endospores; even more preferably the one or more spores are endospores of Bacillus subtilis, Bacillus licheniformis, Bacillus amyloliquefaciens, or Bacillus megaterium.

The microorganisms may be included in the detergent composition or additive in the same way as enzymes (see below).

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Adjunct Materials

Any detergent components known in the art for use in laundry/ADW/hard surface cleaning detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry/ADW/hard surface cleaning detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Microorganisms

The detergent composition as described above may comprise one or more microorganisms or microbes. Generally, any microorganism(s) may be used in the enzyme/detergent formulations in any suitable amount(s)/concentration(s). Microorganisms may be used as the only biologically active ingredient, but they may also be used in conjunction with one or more of the enzymes described above.

The purpose of adding the microorganism(s) may, for example, be to reduce malodor as described in WO 2012/112718. Other purposes could include in-situ production of desirable biological compounds, or inoculation/population of a locus with the microorganism(s) to competitively prevent other non-desirable microorganisms form populating the same locus (competitive exclusion).

The term “microorganism” generally means small organisms that are visible through a microscope. Microorganisms often exist as single cells or as colonies of cells. Some microorganisms may be multicellular. Microorganisms include prokaryotic (e.g., bacteria and archaea) and eurkaryotic (e.g., some fungi, algae, protozoa) organisms. Examples of bacteria may be Gram-positive bacteria or Gram-negative bacteria. Example forms of bacteria include vegetative cells and endospores. Examples of fungi may be yeasts, molds and mushrooms. Example forms of fungi include hyphae and spores. Herein, viruses may be considered microorganisms.

Microorganisms may be recombinant or non-recombinant. In some examples, the microorganisms may produce various substances (e.g., enzymes) that are useful for inclusion in detergent compositions. Extracts from microorganisms or fractions from the extracts may be used in the detergents. Media in which microorganisms are cultivated, or extracts or fractions from the media may also be used in detergents. In some examples, specific of the microorganisms, substances produced by the microorganisms, extracts, media, and fractions thereof, may be specifically excluded from the detergents. In some examples, the microorganisms, or substances produced by, or extracted from, the microorganisms, may activate, enhance, preserve, prolong, and the like, detergent activity or components contained with detergents.

Generally, microorganisms may be cultivated using methods known in the art. The microorganisms may then be processed or formulated in various ways. In some examples, the microorganisms may be desiccated (e.g., lyophilized). In some examples, the microorganisms may be encapsulated (e.g., spray drying). Many other treatments or formulations are possible. These treatments or preparations may facilitate retention of microorganism viability over time and/or in the presence of detergent components. In some examples, however, microorganisms in detergents may not be viable. The processed/formulated microorganisms may be added to detergents prior to, or at the time the detergents are used.

In one embodiment, the microorganism is a species of Bacillus, for example, at least one species of Bacillus selected from the group consisting of Bacillus subtilis, Bacillus amyloliquefaciens, Bacillus licheniformis, Bacillus atrophaeus, Bacillus pumilus, Bacillus megaterium, or a combination thereof. In a preferred embodiment, the aforementioned Bacillus species are on an endospore form, which significantly improves the storage stability.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. They can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The alpha-amylases of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, alpha-amylases, optionally one or more additional enzymes, a protease inhibitor, and a salt of a monovalent cation and an organic anion may be prepared and the mixture is then plodded. The alpha-amylases and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, and WO09/015951.

WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905,

WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792,

WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### Liquid Enzyme Formulations

The enzyme may be formulated as a liquid enzyme formulation, which is generally a pourable composition, though it may also have a high viscosity. The physical appearance and properties of a liquid enzyme formulation may vary a lot—for example, they may have different viscosities (gel to water-like), be colored, not colored, clear, hazy, and even with solid particles like in slurries and suspensions. The minimum ingredients are the enzyme and a solvent system to make it a liquid. In addition to the enzyme, the liquid enzyme formulation may also comprise other enzyme activities, such as protease, amylase, lipase, cellulase, and/or nuclease (e.g., DNase, RNase) activities.

The solvent system may comprise water, polyols (such as glycerol, (mono, di, or tri) propylene glycol, (mono, di, or tri) ethylene glycol, sugar alcohol (e.g. sorbitol, mannitol, erythritol, dulcitol, inositol, xylitol or adonitol), polypropylene glycol, and/or polyethylene glycol), ethanol, sugars, and salts. Usually the solvent system also includes a preservation agent and/or other stabilizing agents.

A liquid enzyme formulation may be prepared by mixing a solvent system and an enzyme concentrate with a desired degree of purity (or enzyme particles to obtain a slurry/suspension).

In an embodiment, the liquid enzyme composition comprises:

(a) at least 0.01% w/w active enzyme protein,
(b) at least 0.5% w/w polyol,
(c) water, and
(d) optionally a preservation agent.

The enzyme in the liquid composition of the invention may be stabilized using conventional stabilizing agents. Examples of stabilizing agents include, but are not limited to, sugars like glucose, fructose, sucrose, or trehalose; addition of salt to increase the ionic strength; divalent cations (e.g., Ca²⁺ or Mg²⁺); and enzyme inhibitors, enzyme substrates, or various polymers (e.g., PVP). Selecting the optimal pH for the formulation may be very important for enzyme stability. The optimal pH depends on the specific enzyme but is typically in the range of pH 4-9. In some cases, surfactants like nonionic surfactant (e.g., alcohol ethoxylates) can improve the physical stability of the enzyme formulations.

One embodiment of the invention relates to a composition comprising an enzyme, wherein the composition further comprises:

(i) a polyol, preferably selected from glycerol, (mono, di, or tri) propylene glycol, (mono, di, or tri) ethylene glycol, polyethylene glycol, sugar alcohols, sorbitol, mannitol, erythritol, dulcitol, inositol, xylitol and adonitol;
(ii) optionally an additional enzyme, preferably selected from protease, amylase, or lipase,
(iii) optionally a surfactant, preferably selected from anionic and nonionic surfactants,
(iv) optionally a divalent cation, polymer, or enzyme inhibitor;
(v) optionally having a pH in the range of pH 4-9; and
(vi) water.

Slurries or dispersions of enzymes are typically prepared by dispersing small particles of enzymes (e.g., spray-dried particles) in a liquid medium in which the enzyme is sparingly soluble, e.g., a liquid nonionic surfactant or a liquid polyethylene glycol. Powder can also be added to aqueous systems in an amount so not all go into solution (above the solubility limit). Another format is crystal suspensions which can also be aqueous liquids (see for example WO2019/002356). Another way to prepare such dispersion is by preparing water-in-oil emulsions, where the enzyme is in the water phase, and evaporate the water from the droplets. Such slurries/suspension can be physically stabilized (to reduce or avoid sedimentation) by addition of rheology modifiers, such as fumed silica or xanthan gum, typically to get a shear thinning rheology.

### Granular Enzyme Formulations

The enzyme may also be formulated as a solid/granular enzyme formulation. Non-dusting granulates may be produced, e.g. as disclosed in U.S. Pat. Nos. 4,106,991 and 4,661,452, and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The enzyme may be formulated as a granule for example as a co-granule that combines one or more enzymes or benefit agents (such as MnTACN or other bleaching components). Examples of such additional enzymes include proteases, amylases, lipases, cellulases, and/or nucleases (e.g., DNase, RNase). Each enzyme will then be present in more granules securing a more uniform distribution of enzymes in the detergent. This also reduces the physical segregation of different enzymes due to different particle sizes. Methods for producing multi-enzyme co-granulate for the detergent industry are disclosed in the IP.com disclosure IPCOM000200739D.

An embodiment of the invention relates to an enzyme granule/particle comprising a enzyme. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm.

The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation. Methods for preparing the core can be found in Handbook of Powder Technology; Particle size enlargement by C. E. Capes; Volume 1; 1980; Elsevier. These methods are well-known in the art and have also been described in international patent application WO2015/028567, pages 3-5, which is incorporated by reference.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606.

Such coatings are well-known in the art, and have earlier been described in, for example, WO00/01793, WO2001/025412, and WO2015/028567, which are incorporated by reference.

In one aspect, the present invention provides a granule, which comprises:

(a) a core comprising an enzyme according to the invention; and
(b) optionally a (salt) coating consisting of one or more layer(s) surrounding the core.

Another aspect of the invention relates to a layered granule, comprising:

(a) a (non-enzymatic) core;
(b) a coating surrounding the core, wherein the coating comprises an enzyme; and
(c) optionally a (salt) coating consisting of one or more layer(s) surrounding the enzyme containing coating.

### Encapsulated Enzyme Formulations

The enzyme may also be formulated as an encapsulated enzyme formulation (an ‘encapsulate’). This is particularly useful for separating the enzyme from other ingredients when the enzyme is added into, for example, a (liquid) cleaning composition, such as the detergent compositions described below.

Physical separation can be used to solve incompatibility between the enzyme(s) and other components. Incompatibility can arise if the other components are either reactive against the enzyme, or if the other components are substrates of the enzyme. Other enzymes can be substrates of proteases.

The enzyme may be encapsulated in a matrix, preferably a water-soluble or water dispersible matrix (e.g., water-soluble polymer particles), for example as described in WO 2016/023685. An example of a water-soluble polymeric matrix is a matrix composition comprising polyvinyl alcohol. Such compositions are also used for encapsulating detergent compositions in unit-dose formats.

The enzyme may also be encapsulated in core-shell microcapsules, for example as described in WO 2015/144784, or as described in the IP.com disclosure IPCOM000239419D.

Such core-shell capsules can be prepared using a number of technologies known in the art, e.g., by interfacial polymerization using either a water-in-oil or an oil-in-water emulsion, where polymers are crosslinked at the surface of the droplets in the emulsion (the interface between water and oil), thus forming a wall/membrane around each droplet/capsule.

### Methods of Use

The invention provides a use of a detergent composition in a domestic or industrial cleaning process. A cleaning process may for example be a dishwashing process, such as automated dishwashing; a laundry process; or cleaning of hard surfaces such as bathroom tiles, floors, table tops, drains, sinks and washbasins.

Dishwashing

An automated dishwashing process may comprise the following steps:

a. Exposing dishware to an aqueous wash liquor comprising a detergent composition;

b. Completing at least one wash cycle; and

c. Optionally rinsing and drying the dishware.

Thus, the invention provides a method of dishwashing in an automatic dishwashing machine using a detergent composition as described herein, comprising the steps of adding said detergent composition in a detergent composition compartment in said automatic dishwashing machine, and releasing said detergent composition during a main-wash cycle.

The compositions may be employed at concentrations from about 1000-8000 ppm in the wash liquor, such as 2000-6000 ppm in the wash liquor. The hardness of the wash liquor may be 3-30° dH. The pH of the wash liquor may be 3-11, such as 7-11.

The temperature of the wash liquor when used may be in the range of 10−70° C. For example the temperature of the wash liquor can be in the range of 15−60° C., in the range of 20-50° C., in the range of 25−50° C., in the range of 30−45° C., in the range of 35−40° C., in the range of 35−55° C., or in the range of 40−50° C.

The temperature may vary throughout the wash program. One enzyme may be activated at one active temperature range and other enzymes may be activated at another active temperature range differing from the active temperature range of the first enzyme. For example, one or more wash cycles may be carried out at a temperature of 32-38° C. and other wash cycles may be carried out at a temperature of 45-55° C. The advantage of this is that the single enzymes are allowed to work at their optimal temperature. The optimal temperature of the enzymes of a detergent composition may vary but is typically in the range of 65−70° C. for proteases and in the range of 55−65° C. for amylases. The optimal temperature may be determined by different assays, such as comparing the activity over a 15 min period of time in a buffered solution at different temperatures.

During or after completion of a wash cycle the dishware can be rinsed with water or with water comprising a rinsing aid. The effectiveness of the cleaning can be further improved if an acidic rinsing aid is used. The rinsing aid should be capable of lowering the pH below 4 during at least a period of the rinsing step. The pH may be even further lowered e.g. to below pH 3.5, such as below pH 3, below pH 2.5 or below pH 2. The period of lowering the pH may be at least 1 minute, such as at least 2 minutes, at least 3 minutes, at least 4 minutes, at least 5 minutes, at least 6 minutes or at least 7 minutes. The period of lowering the pH may even be as long as the time period for the full rinsing step.

The ability of lowering the pH during the rinsing step is due to a buffering agent. A buffer with strong buffer capacity at low pH, from pH 4 and below should be selected. The buffer capacity should correspond to the same effect as the pH drop was done with 15 ml 4M HCL/rinse cycle. The ability of lowering the pH during the rinsing step is due to a buffering agent selected from the group consisting of citric acid, acetic acid, potassium dihydrogen phosphate, boric acid, diethyl barbituric acid, Carmody buffer and Britton-Robinson buffer.

The rinsing aid can further improve the cleaning of the dishware by rinsing away any soil released from the dishware during the washing cycle. In addition, the acidic rinsing aid prevents precipitation of calcium on the dishware.

Laundering

Laundry processes can for example be household laundering, but it may also be industrial laundering. A process for laundering of fabrics and/or garments may be a process comprises treating fabrics with a washing solution containing a detergent composition as described herein. A cleaning process or a textile care process can for example be carried out in a machine washing process or in a manual washing process.

The fabrics and/or garments subjected to a washing, cleaning or textile care process may be conventional washable laundry, for example household laundry. Preferably, the major part of the laundry is garments and fabrics, including knits, woven, denims, non-woven, felts, yarns, and toweling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibres (tricell), lyocell or blends thereof. The fabrics may also be non-cellulose based such as natural polyamides including wool, camel, cashmere, mohair, rabbit and silk or synthetic polymer such as nylon, aramid, polyester, acrylic, polypropylene and spandex/elastane, or blends thereof as well as blend of cellulose based and non-cellulose based fibres.

In one aspect, the present invention relates to a method of laundering in an automatic laundering machine using a detergent composition as described herein, comprising the steps of adding said detergent composition in a detergent composition compartment in said automatic laundering machine, and releasing said detergent composition during a main wash cycle. In another aspect, the present invention relates to a method of laundering, comprising laundering a garment with a detergent composition as described herein, preferably at a temperature of 50° C. or less, or more preferably at a temperature of 45° C. or less, or even more preferably at a temperature of 40° C. or less even more preferably at a temperature of 35° C. or less or even more preferably at a temperature of 30° C. or less, even more preferably at a temperature of 25° C. or less or even more preferably at a temperature of 20° C. or less.

These methods include a method for laundering a fabric. The method comprises the steps of contacting a fabric to be laundered with a cleaning laundry solution comprising a detergent composition. The fabric may comprise any fabric capable of being laundered in normal consumer use conditions. The solution preferably has a pH from about 5.5 to about 11.5. The compositions may be employed at concentrations from about 100 ppm, preferably 500 ppm to about 15,000 ppm in solution. The water temperatures typically range from about 5° C. to about 95° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a degree of hardness of from about 0° dH to about 30° dH. Under typical European wash conditions, the degree of hardness is about 16° dH, under typical US wash conditions about 6° dH, and under typical Asian wash conditions, about 3° dH.

### Uses

The present invention further relates to the use of detergent composition according to the present invention in a cleaning process such as laundry, including industrial cleaning, ADW and hard surface cleaning. The soils and stains that are important for cleaning are composed of many different substances, and a range of different enzymes, all with different substrate specificities, have been developed for use in detergents both in relation to laundry and hard surface cleaning, such as dishwashing. These enzymes are considered to provide an enzyme detergency benefit, since they specifically improve stain removal in the cleaning process that they are used in, compared to the same process without enzymes. Stain removing enzymes that are known in the art include enzymes such as proteases, amylases, lipases, cutinases, cellulases, endoglucanases, xyloglucanases, pectinases, pectin lyases, xanthanases, peroxidaes, haloperoxygenases, catalases and mannanases.

In another aspect, the invention relates to a laundering process which may be for household laundering as well as industrial laundering. Furthermore, the invention relates to a process for the laundering of textiles (e.g. fabrics, garments, cloths etc.) where the process comprises treating the textile with a washing solution containing a detergent composition of the present invention. The laundering can for example be carried out using a household or an industrial washing machine or be carried out by hand using a detergent composition of the invention.

In another aspect, the invention relates to a dish wash process, including ADW; or hard surface cleaning, which may be for household cleaning as well as industrial cleaning. Furthermore, the invention relates to a process for dish wash or hard surface cleaning, where the process comprises treating the dishes or hard surfaces with a washing solution comprising a detergent composition of the present invention. The dish wash or hard surface cleaning can for example be carried out using a household dish washing machine or be carried out by hand using a detergent composition of the invention.

The present invention is further described by the following examples that should not be construed as limiting the scope of the invention.

### Materials and Method

### Assays for Alpha-Amylase Activity

### 1. PNP-G7 Assay

The alpha-amylase activity was determined by employing the pNP-G7 substrate (PNP-G7 the abbreviation for 4,6-ethylidene(G7)-p-nitrophenyl(G1)-α,D-maltoheptaoside, a blocked oligosaccharide which is cleaved by an endo-amylase, such as an alpha-amylase).

An antibody was diluted in Phosphate buffered saline (PBS) (0.010 M Phosphate buffer pH7.4, 0.0027M KCl, 0.14M NaCl) buffer to concentration of 10 μg/ml. A maxisorp microtiter plate was coated with antibody by adding 100 μl diluted antibody (10 μg/ml) to each well and incubated for 1 h at room temperature (RT) and mixing at 800 rpm. After incubation the microtiter plate was washed (using Bio-Tek ELx405 ELISA washer) with 3×200 μl Phosphate buffered saline with 0.05% Tween (PBST) (0.010 M Phosphate buffer pH7.4, 0.0027M KCl, 0.14M NaCl, 0.05% Tween 20) buffer.

Microtiter plates with the alpha-amylase variants culture broths were spun down and supernatants transferred to new microtiter plates and diluted 4× in PBST buffer. 100 μl diluted supernatant was transferred to antibody coated maxisorp microtiter plate and incubated for 1 h at RT and mixing at 800 rpm. After incubation microtiter plates were washed in PBST buffer (3×200 μl, ELISA washer).

Upon the cleavage of the pNP-G7 substrate, the alpha-Glucosidase included in the kit used is digested and the hydrolysed substrate liberates a free pNP molecule which has a yellow color and thus can be measured by visible spectophometry at Abs=405 nm (400-420 nm.). Kits containing pNP-G7 substrate and alpha-Glucosidase are manufactured by Roche/Hitachi (cat. No. 11876473). 100 μl pNP-G7 substrate was added to all wells and mixed for 1 minute before measuring absorbance at 405 nm. The slope (absorbance per minute) is determined and only the linear range of curve is used.

The slope of the time dependent absorption-curve is directly proportional to the activity of the alpha-amylase in question under the given set of conditions.

The specific alpha-amylase activity may also be determined by other activity assays, such as amylazyme activity assay, Phadebas activity assay, or reducing sugar activity assay as described below.

### 2. Amylazyme Activity Assay

Amylazyme activity assay (from Megazyme, Ireland): An Amylazyme tablet includes interlinked amylose polymers that are in the form of globular microspheres that are insoluble in water. A blue dye is covalently bound to these microspheres. The interlinked amylose polymers in the microsphere are degraded at a speed that is proportional to the alpha-amylase activity. When the alpha-amylase degrades the amylose polymers, the released blue dye is water soluble and concentration of dye can be determined by measuring absorbance at 650 nm. The concentration of blue is proportional to the alpha-amylase activity in the sample.

The amylase sample to be analysed is diluted in activity buffer with the desired pH. One substrate tablet is suspended in 5 mL activity buffer and mixed on magnetic stirrer. During mixing of substrate transfer 150 μl to microtiter plate (MTP). Add 30 μl diluted amylase sample to 150 μl substrate and mix. Incubate for 15 minutes at 37° C. The reaction is stopped by adding 30 μl 1M NaOH and mix. Centrifuge MTP for 5 minutes at 4000×g. Transfer 100 μl to new MTP and measure absorbance at 620 nm.

The amylase sample should be diluted so that the absorbance at 650 nm is between 0 and 2.2, and is within the linear range of the activity assay.

### 3. Phadebas Assay

A Phadebas tablet (from for example Magle Life Sciences, Lund, Sweden) includes interlinked starch polymers that are in the form of globular microspheres that are insoluble in water. A blue dye is covalently bound to these microspheres. The interlinked starch polymers in the microsphere are degraded at a speed that is proportional to the alpha-amylase activity. When the alpha-amylase degrades the starch polymers, the released blue dye is water soluble and concentration of dye can be determined by measuring absorbance at 650 nm. The concentration of blue is proportional to the alpha-amylase activity in the sample.

The amylase sample to be analysed is diluted in activity buffer with the desired pH. One substrate tablet is suspended in 5 mL activity buffer and mixed on magnetic stirrer. During mixing of substrate transfer 150 μl to microtiter plate (MTP). Add 30 μl diluted amylase sample to 150 μl substrate and mix. Incubate for 15 minutes at 37° C. The reaction is stopped by adding 30 μl 1M NaOH and mix. Centrifuge MTP for 5 minutes at 4000×g. Transfer 100 μl to new MTP and measure absorbance at 620 nm.

The measured absorbance is directly proportional to the specific activity (activity/mg of pure alpha-amylase protein) of the alpha-amylase in question under the given set of conditions.

### 4. Reducing Sugar Activity Assay

Number of reducing ends formed by the alpha-amylase hydrolysing the alpha-1,4-glycosidic linkages in starch is determined by reaction with p-Hydroxybenzoic acid hydrazide (PHBAH). After reaction with PHBAH the number of reducing ends can be measured by absorbance at 405 nm and the concentration of reducing ends is proportional to the alpha-amylase activity in the sample.

The corns starch substrate (3 mg/ml) is solubilised by cooking for 5 minutes in milliQ water and cooled down before assay. For the stop solution prepare a Ka-Na-tartrate/NaOH solution (K—Na-tartrate (Merck 8087) 50 g/I, NaOH 20 g/1) and prepare freshly the stop solution by adding p-Hydroxybenzoic acid hydrazide (PHBAH, Sigma H9882) to Ka-Na-tartrate/NaOH solution to 15 mg/ml.

In PCR-MTP 50 μl activity buffer is mixed with 50 μl substrate. Add 50 μl diluted enzyme and mix. Incubate at the desired temperature in PCR machine for 5 minutes. Reaction is stopped by adding 75 μl stop solution (Ka-Na-tartrate/NaOH/PHBAH). Incubate in PCR machine for 10 minutes at 95° C. Transfer 150 μl to new MTP and measure absorbance at 405 nm.

The measured absorbance is directly proportional to the specific activity (activity/mg of pure alpha-amylase protein) of the alpha-amylase in question under the given set of conditions.

### Assessment of Wash Performance of Alpha-Amylase Variants Using Automatic Mechanical Stress Assay (AMSA) for Automatic Dish Wash

Washing experiments are performed in order to assess the wash performance of the polypeptides of the present invention in a detergent base composition tested using the Automatic Mechanical Stress Assay (AMSA). With the AMSA, the wash performance of many small volume enzyme-detergent solutions can be examined. The AMSA plate has a number of slots for test solutions and a lid that firmly squeezes the dish wash monitor to be washed against the slot openings. During the wash, the plate, test solutions, dishwash monitor and lid are vigorously shaken to bring the test solution in contact with the soiled dishwash monitor and apply mechanical stress in a regular, periodic oscillating manner. For further description see WO 02/42740 especially the paragraph “Special method embodiments” at page 23-24.

The experiment may be conducted under the experimental conditions as specified.

Water hardness was adjusted to 21° dH by addition of CaCl₂, MgCl₂, and NaHCO₃ (Ca²⁺:Mg²⁺:HCO₃⁻=4:1:10) to Milli-Q water.

The wash performance is measured as the brightness of the color of the dish wash monitor. Brightness can also be expressed as the intensity of the light reflected from the sample when illuminated with white light. When the sample is stained the intensity of the reflected light is lower, than that of a clean sample. Therefore, the intensity of the reflected light can be used to measure wash performance.

Color measurements are made with a professional flatbed scanner, which is used to capture an image of the washed textile. To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

In these tests, different amylase was tested; each of the amylases were tested alone and in combination. The “expected additive effect” is the light remission expected if the two amylase act additively. The “synergy” is the actual light remission measured when the two amylase are combined, minus the “expected additive effect”. The “synergy” therefore reflects the increase in wash performance that the combination of enzymes achieves compared to the additive effects of their individual performance.

### Assessment of Wash Performance of Alpha-Amylase Variants Using Automatic Dish Wash (ADW)

In order to assess the wash performance of the alpha-amylase variants of the present invention in a detergent base composition, washing experiments may be performed using full scale Automatic Dish Wash (ADW). The full scale ADW setup is used for testing the wash performance of polypeptides in test conditions mimicking a regular consumer setup. The full-scale wash performance experiments were conducted under the experimental conditions.

In these tests, different amylase was tested; each of the amylases were tested alone and in combination. The “expected additive effect” is the light remission expected if the two amylase act additively. The “synergy” is the actual light remission measured when the two amylase are combined, minus the “expected additive effect”. The “synergy” therefore reflects the increase in wash performance that the combination of enzymes achieves compared to the additive effects of their individual performance.

1. A detergent composition comprising an amylase, wherein said amylase is a mixture of at least two amylases.

2. The composition according to claim 1, wherein the amylase is selected from a group consisting of:
a. variant of a parent amylase, said variant amylase or parent amylase has at least 60% sequence identity to SEQ ID NO: 1 and further comprising a mutation at one or more positions corresponding to position 9, 26, 30, 33, 37, 74, 82, 106, 118, 128, 133, 149, 150, 160, 178, 182, 186, 190, 193, 195, 202, 203, 214, 231, 256, 257, 258, 269, 270, 272, 283, 295, 296, 298, 299, 303, 304, 305, 311, 314, 315, 318, 319, 320, 323, 339, 345, 361, 365, 378, 383, 419, 421, 437, 441, 444, 445, 446, 447, 450, 458, 461, 471, 482, or 484, and optionally at least one mutation at an amino acid corresponding to 181, 182, 183 and 184 (using SEQ ID NO: 1 for numbering);
b. variant of a parent amylase, said variant amylase or parent amylase has at least 60% sequence identity to SEQ ID NO: 2 and further comprising a mutation at one or more positions corresponding to position: 51, 246 or 334 and the position 202 comprises a methionine (using SEQ ID NO: 2 for numbering).
c. variant of a parent amylase, said variant amylase or parent amylase has at least 60% sequence identity to SEQ ID NO: 3 and optionally at least one mutation at an amino acid corresponding to 181, 182, 183 and 184 (using SEQ ID NO: 3 for numbering);
d. variant of a parent amylase, said variant amylase or parent amylase has at least 60% sequence identity to SEQ ID NO: 4 and further comprising a mutation at one or more positions corresponding to position 1, 7, 9, 11, 16, 19, 25, 37, 43, 48, 54, 56, 58, 59, 60, 63, 74, 81, 84, 86, 90, 98, 104, 109, 111, 113, 116, 118, 125, 127, 130, 132, 133, 134, 135, 136, 139, 142, 144, 149, 158, 160, 163, 167, 169, 170, 171, 172, 173, 174, 175, 176, 178, 181, 182, 186, 187, 190, 195, 202, 203, 204, 206, 209, 210, 212, 227, 235, 238, 246, 256, 259, 264, 265, 266, 267, 269, 270, 272, 273, 274, 275, 276, 284, 286, 291, 293, 295, 298, 299, 302, 303, 304, 306, 310, 311, 314, 315, 317, 319, 320, 323, 328, 337, 339, 345, 357, 365, 377, 375, 385, 391, 395, 400, 406, 408, 410, 431, 435, 439, 444, 445, 458, 465, 466, 469, 473, 476 or 481 and optionally at least one mutation at an amino acid corresponding to 181, 182, 183 and 184 (using SEQ ID NO: 4 for numbering);
e. variant of a parent amylase, said variant amylase or parent amylase has at least 60% sequence identity to SEQ ID NO: 5 and further comprising a mutation at one or more positions corresponding to position: 405, 421, 422, or 428 (using SEQ ID NO: 5 for numbering);
f. variant of a parent amylase, said variant amylase or parent amylase has at least 60% sequence identity to SEQ ID NO: 6 and further comprising a mutation at one or more positions corresponding to position: 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 and optionally at least one mutation at an amino acid corresponding to 181, 182, 183 and 184 (using SEQ ID NO: 6 for numbering);
g. a variant of a parent amylase, said variant amylase or parent amylase has at least 60% sequence identity to SEQ ID NO: 7 and further comprising a mutation at one or more positions corresponding to position: 87, 98, 125, 128, 131, 165, 178, 201, 202, 225, 243, 272, 282, 305, 309, 319, 320, 359, 444, or 475, and optionally at least one mutation at an amino acid corresponding to 181, 182, 183 and 184 (using SEQ ID NO: 7 for numbering),
h. a variant of a parent amylase, said variant amylase or parent amylase has at least 60% sequence identity to SEQ ID NO: 8 and further comprising a mutation at one or more positions corresponding to position: 38, 88, 92, 126, 129, 134, 153, 171, 187, 203, 241, 275, 303, 360, 375, 458, 459, 460, 476, or 477, and optionally at least one mutation at an amino acid corresponding to 178, 179, 180, 181 (using SEQ ID NO: 8 for numbering),
i. a variant of a parent amylase, said variant amylase or parent amylase has at least 60% sequence identity to SEQ ID NO: 9 and further comprising a mutation at one or more positions corresponding to position: 1, 7, 109, 140, 195, 206, 243, 260, 280, 284, 304, 320, 323, 391 and 476, and optionally at least one mutation at an amino acid corresponding to 180, 181, 183, 184 (using SEQ ID NO: 9 for numbering), and
j. variant of a parent amylase, said variant amylase or parent amylase has at least 60% sequence identity to SEQ ID NO: 10 and further comprising a mutation at one or more positions corresponding to position 74, 172, 190, 202, 208, 255, 261, or 365 (using SEQ ID NO: 10 for numbering).

3. The composition according to claim 1 further comprising one or more additional active components.

4. The composition according to claim 3, wherein one or more additional active component is selected from the group consisting of one or more enzymes, oxidizing agents, bleach activators, bleach catalysts, chelating agents, bulking agents, builders, buffering agents, structurants, sequestrants, optical brighteners, antifoaming agents, fragrances, anti-redeposition agents, crystal growth inhibitors, metal care agents, glass care agents, skin conditioning agents, softness extenders, emulsifiers, and colorants.

5. The composition according to claim 4, wherein the one or more enzymes is selected from the group consisting of proteases, amylases, phospho-lipases, esterases, lipases, cutinases, cellulases, endoglucanases, xyloglucanases, xylanases, pectinases, hemicellulases pectin lyases, xanthanases, peroxidases, keratinases haloperoxygenases, catalases, mannanases, lechinase, RNase, DNAse, reductases, oxidases, phenoloxidases, lipoxygenases, ligninases, pullulanases, tannases, pento-sanases, malanases, β-glucanases, arabinosidases, hyaluronidase, chondroitinase, and laccase or any mixture thereof.

6. The composition according to claim 4, wherein the surfactant is one or more surfactants selected from the group consisting of an anionic surfactant, a cationic surfactant, a non-ionic surfactant, zwitterionic surfactant, and amphoteric surfactants or any mixtures thereof.

7. The composition according to claim 1, wherein the composition comprises amylase in the range from 0.001 to 10 mg per gram of the composition.

8. The composition according to claim 1, wherein the composition has a pH in the range of 2.0-12.0.

9. The composition according to claim 1, wherein the composition has improved performance as compared to said reference alpha-amylase.

10. The composition according to claim 1, wherein the detergent composition is a liquid or powder laundry detergent composition.

11. The composition according to claim 1, wherein the detergent composition is a liquid or powder automatic dishwashing (ADW) detergent composition, wherein said composition is a hand or machine laundry detergent composition.

12. A method of automatic dishwashing comprising the following steps: a) providing soiled dishware; b) placing the dishware into an automatic dishwasher; c) providing an automatic dishwashing cleaning composition according to claim 1; and d) subjecting the dishware to a wash cycle of equal or less than 60 mins.

13. A method of automatic dishwashing comprising the following steps: a) providing soiled dishware; b) placing the soiled dishware into an automatic dishwasher; c) providing an automatic dishwashing cleaning composition according to claim 1; and d) subjecting the dishware to a wash cycle at a temperature of 80 degrees centigrade or below.

14. A method for removing a stain from a surface comprising contacting the surface with a composition according to claim 1.

15. (canceled)
